(19) United States
(12) Patent Application Publication
(10) Pub. No.: US 2021/0219850 A1
(43) Pub. Date: Jul. 22, 2021

(54) PROVIDING A BLOOD FLOW PARAMETER SET FOR A VASCULAR MALFORMATION

(71) Applicant: Siemens Healthcare GmbH, Erlangen (DE)

(72) Inventor: Annette Birkhold, Nürnberg (DE)

(21) Appl. No.: 17/151,116

(22) Filed: Jan. 15, 2021

(30) Foreign Application Priority Data
Jan. 22, 2020 (DE) 10 2020 200 750.0

(51) Int. Cl. A61B 5/026; A61B 5/107; A61B 5/00; G06T 7/00

(52) U.S. Cl. CPC A61B 5/026; A61B 5/107; G06T 2207/30104; G06T 7/0016; G06T 2207/20081; A61B 5/7267

(57) ABSTRACT

A computer-implemented method for providing a blood flow parameter set for a vascular malformation includes receiving time-resolved image data. The image data maps a change over time in a vessel section of an examination subject. The vessel section includes the vascular malformation. A time-resolved image of the vessel section is reconstructed from the image data. The vascular malformation is segmented in the image of the vessel section. An afferent and an efferent vessel are identified at the vascular malformation based on the image of the vessel section. An average blood flow velocity parameter and a vessel cross-sectional area parameter are determined for each of the afferent and the efferent vessel. The method includes determining and providing the blood flow parameter set for the vascular malformation based on the average blood flow velocity parameters and the vessel cross-sectional area parameters.

This application claims the benefit of German Patent Application No. 10 2020 200 750.0, filed on Jan. 22, 2020, which is hereby incorporated by reference in its entirety.

### BACKGROUND

The present embodiments relate to a computer-implemented method for providing a blood flow parameter set for a vascular malformation, a computer-implemented method for providing a trained function, a provider unit, a training unit, a medical imaging device, a computer program product, and a computer-readable storage medium.

A precise knowledge of all vessels adjoining a vascular malformation is often a prerequisite for a diagnosis and/or treatment of vascular malformations as a form of vascular lesions. A vascular malformation often connects an arterial vascular system (e.g., having a high pressure) to a venous vascular system (e.g., having a low pressure). It is therefore often of importance for a good treatment outcome to determine the pressure ratios at least at the interfaces of the vascular malformation with the adjoining arterial and venous vessels to the best possible extent. Ruptures and/or bleeding may occur as a result of incorrect estimations of the pressure ratios.

For this reason, the blood flow in aneurysms as a manifestation of vascular malformations is frequently estimated based on optical flow principles through a combination of image data of a 3D digital rotational angiography scan (3DRA) and a 2D digital subtraction angiography scan (2D DSA). However, a disadvantage in this case is that this form of blood flow estimation has only limited applicability due to the complex geometry of arteriovenous malformations (AVMs).

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a particularly reliable, imaging-based determination of a blood flow parameter set for a vascular malformation is enabled.

The description hereinbelow not only relates to methods and devices for providing a blood flow parameter set for a vascular malformation, but also relates to methods and devices for providing a trained function. Features, advantages, and alternative embodiment variants of data structures and/or functions in methods and devices for providing a blood flow parameter set for a vascular malformation may herein be applied to analogous data structures and/or functions in methods and devices for providing a trained function. Analogous data structures may, in this case, be characterized, for example, through the use of the prefix “training”. Further, the trained functions used in methods and devices for providing a blood flow parameter set for a vascular malformation may have been adapted and/or provided, for example, by methods and devices for providing a trained function.

The present embodiments relate in a first aspect to a computer-implemented method for providing a blood flow parameter set for a vascular malformation including multiple acts. Here, in a first act a), time-resolved image data is received. The image data maps a change over time in a vessel section of an examination subject. In this case, the vessel section includes the vascular malformation. In a second act b), a time-resolved image of the vessel section is reconstructed from the image data. In a third act c), the vascular malformation is segmented in the image of the vessel section. After this, in act d1), at least one afferent vessel at the vascular malformation is identified based on the image of the vessel section. Further, in act d2), at least one efferent vessel at the vascular malformation is identified based on the image of the vessel section. In a further act e1), an average blood flow velocity parameter is determined in each case for the at least one afferent vessel and the at least one efferent vessel. Further, in act e2), a vessel cross-sectional area parameter is determined in each case for the at least one afferent vessel and the at least one efferent vessel. In this process, acts c), d1), and/or d2) may be performed in any order with respect to one another and/or concurrently. Similarly, acts e1) and e2) may, for example, be performed sequentially and/or concurrently. Also, in act f1), the blood flow parameter set for the vascular malformation is determined based on the average blood flow velocity parameters and the vessel cross-sectional area parameters. The blood flow parameter set is provided in a further act g).

The receiving of the time-resolved image data in act a) may, for example, include an acquisition and/or a readout of a computer-readable data storage medium and/or a receiving from a data storage unit (e.g., a database). Time-resolved image data may further be provided by a provider unit of a medical imaging device.

Further, the time-resolved image data may include multiple picture elements (e.g., pixels and/or voxels). In one embodiment, the time-resolved image data at least partially images a common vessel section of the examination subject. In this case, the time-resolved image data may include two-dimensional and/or three-dimensional images of the vessel section that have been acquired in chronological sequence. In this case, the image data may, for example, include two-dimensional projection X-ray images and/or three-dimensional computed tomography data. In one embodiment, the image data may have been acquired from different projection directions (e.g., angulations) with respect to the vessel section of the examination subject. Further, the image data may include metadata. The metadata may include, for example, information relating to an acquisition parameter and/or operating parameter of the medical imaging device.

Further, the image data may map a change over time (e.g., a propagation movement and/or flow movement of a contrast medium in the vessel section of the examination subject and/or a movement of a medical object, such as a guide wire and/or a catheter and/or an endoscope and/or a laparoscope) in the vessel section of the examination subject. In this case, the examination subject may be a human and/or animal patient, for example.

In one embodiment, the vessel section includes the vascular malformation. In this case, the vascular malformation may be embodied, for example, as a vascular lesion (e.g., as an arteriovenous malformation (AVM)). Further, the vascular malformation may include a nidus. In this case, the vessel section may also include at least one afferent vessel. The at least one afferent vessel has a blood flow directed toward the vascular malformation. Further, the vessel section may include at least one efferent vessel. The at least one efferent vessel has a blood flow directed away from the vascular malformation.

The reconstruction of the time-resolved image of the vessel section in act b) may, for example, include a Radon transform and/or Fourier transform and/or a backprojection (e.g., a multiplicative backprojection) of the image data. In one embodiment, the time-resolved image of the vessel section may include a plurality of three-dimensional image datasets and associated time information in each case. In this case, the multiple three-dimensional image datasets may be reconstructed from the two-dimensional and/or three-dimensional image data. Further, the multiple three-dimensional image datasets may in each case include multiple picture elements. Time information, in each case, is assigned to at least some of the picture elements. In this case, the time information may, for example, describe an acquisition time at which the image data corresponding to the respective picture element was acquired. For this purpose, the reconstruction may be based, in addition, on the metadata of the time-resolved image data.

The segmenting of the vascular malformation in the image of the vessel section in act c) may, for example, be carried out based on artificial intelligence and/or by, for example, manual and/or semiautomatic annotation and/or based on image values. In this case, the vascular malformation may, for example, be identified and segmented based on a shape in the image of the vessel section. Further, the vascular malformation may be segmented based on a comparison of image values of the image of the vessel section with a predefined threshold value. For example, the segmentation of the vascular malformation may be performed based on image contrast information. As a result of the segmentation of the vascular malformation in the image of the vessel section, the picture elements corresponding to the image of the vascular malformation may be identified and segmented. For example, the picture elements corresponding to the image of the vascular malformation may be annotated and/or marked and/or masked.

The identifying of the at least one afferent vessel at the vascular malformation in act d1) and/or of the at least one efferent vessel at the vascular malformation in act d2) may include an annotation and/or marking and/or localization of picture elements in the image of the vessel section that correspond to an image of the at least one afferent vessel at the vascular malformation and/or to an image of the at least one efferent vessel at the vascular malformation. In this case, the at least one afferent or efferent vessel having a blood flow and/or contrast medium flow directed toward the vascular malformation or away from the vascular malformation respectively may be identified. In this case, the at least one afferent or efferent vessel may adjoin the vascular malformation such that the at least one afferent or efferent vessel has a common cross-sectional area with the vascular malformation. For example, the at least one afferent or efferent vessel may be identified based on the time information associated with each of the three-dimensional image datasets of the image of the vessel section. In this case, a temporal and/or spatial change in image values in the image of the vessel section may be evaluated in order to identify the at least one afferent or efferent vessel at the vascular malformation. Further, a centerline may be determined in each case for the at least one afferent or efferent vessel, where a spatial direction of the change over time in the image values may be determined along the respective centerline. In this case, the respective vessel at the vascular malformation may be identified as the at least one afferent or as the at least one efferent vessel based on the direction of the change over time in the image values. In this case, the at least one afferent vessel may have a blood flow and/or contrast medium flow directed toward the vascular malformation. Further, the at least one efferent vessel may have a blood flow and/or contrast medium flow directed away from the vascular malformation.

In act e1), an average blood flow velocity parameter may be determined in each case for the at least one afferent vessel and the at least one efferent vessel. In this case, the average blood flow velocity parameter may, for example, include information on the time-averaged blood flow velocity of the at least one afferent vessel and/or of the at least one efferent vessel. The average blood flow velocity parameter may be determined, for example, based on the temporal and spatial change in the image values of the image of the vessel section (e.g., along the respective centerline corresponding to the image of the at least one afferent or efferent vessel). Further, the determination of the average blood flow velocity parameter may include the generation of a flow map (e.g., based on a blood flow simulation (computational fluid dynamics (CFD)) and/or the determination of a fast Fourier transform (FFT) based on the image of the vessel section.

The respective one vessel cross-sectional area parameter may, for example, include a spatial measure relating to the vessel cross-sectional area (CSA) (e.g., a radius and/or diameter and/or a cross-sectional area) associated with the at least one afferent vessel and the at least one efferent vessel. Further, the vessel cross-sectional area parameters may be determined, for example, based on anatomical and/or geometric features in the image of the vessel section. For example, the vessel cross-sectional area parameters may be determined based on a spatial distance between picture elements corresponding to an image of a vessel wall of the afferent or efferent vessel. For example, the vessel cross-sectional area parameter may include a vessel cross-sectional area averaged over a segment of the afferent or efferent vessel depicted in the image of the vessel section. Further, the vessel cross-sectional area parameter may include a spatial measure relating to the vessel cross-sectional area at the common cross-sectional area of the vascular malformation with the afferent or efferent vessel.

In act f1), the blood flow parameter set for the vascular malformation may be determined based on the average blood flow velocity parameters and the vessel cross-sectional area parameters. In this case, the blood flow parameter set may, for example, include a blood flow parameter (e.g., a hemodynamic parameter) relating to the at least one afferent vessel and/or the at least one efferent vessel. Further, the blood flow parameter set may, for example, include information on the volume flow rate (e.g., volume flow ratio (VFR)) relating to the at least one afferent vessel and/or the at least one efferent vessel. In this case, one of the blood flow parameters in each case may be determined based on the average blood flow velocity parameter and the vessel cross-sectional area parameter of the respective afferent or efferent vessel (e.g., as a product and/or sum).

Further, the providing of the blood flow parameter set in act g) may, for example, include a storing on a computer-readable storage medium and/or a displaying on a visualization unit and/or a transferring to a provider unit. For example, a graphical visualization of the blood flow parameter set (e.g., an overlaid representation with the image of the vessel section) may be displayed on the visualization unit.

The method of one or more of the present embodiments enables a quantitative determination of blood flow parameters (e.g., a volume flow rate) based on the time-resolved image data. By providing the blood flow parameter set (e.g., a graphical representation of the blood flow parameter set), it is possible to support medical staff in the treatment of embolizations within the imaged vessel section.

In a further embodiment variant of the computer-implemented method for providing a blood flow parameter set for a vascular malformation, the blood flow parameter set may include at least one first blood flow parameter that corresponds to the at least one afferent vessel. In this case, the blood flow parameter set may include at least one second blood flow parameter that corresponds to the at least one efferent vessel. Further, the computer-implemented method of one or more of the present embodiments may also include an act f2), in which a sum of the at least one first blood flow parameter is compared with a sum of the at least one second blood flow parameter. Further, the computer-implemented method of one or more of the present embodiments may be carried out repeatedly as of a predetermined discrepancy between the sums, starting at act d1).

In this case, the comparison in act f2) may, for example, include a difference and/or a quotient between the sum of the at least one first blood flow parameter and the sum of the at least one second blood flow parameter. By the comparison between the sum of the at least one first blood flow parameter and the sum of the at least one second blood flow parameter in act f2), it may be provided that all afferent and/or efferent vessels at the vascular malformation have been identified in acts d1) and d2). Given a predetermined discrepancy between the sum of the at least one first blood flow parameter and the sum of the at least one second blood flow parameter, the method may, for example, be carried out repeatedly starting at act d1), in which case the at least one thus far not identified afferent and/or efferent vessel may be identified. The predetermined discrepancy may, for example, be specified as a function of an accuracy in the determination of the at least one first blood flow parameter and of the at least one second blood flow parameter (e.g., an accuracy in the determination of the average blood flow velocity parameters and/or of the vessel cross-sectional area parameters). Further, the predetermined discrepancy may include a threshold value. The method is carried out repeatedly starting at act d1) in the event of a deviation between the sum of the at least one first blood flow parameter and the sum of the at least one second blood flow parameter above the threshold value.

This enables a validity check to be implemented during the determination of the blood flow parameter set (e.g., during the identification of the at least one afferent vessel and of the at least one efferent vessel). A higher accuracy and improved reliability of the proposed method may be achieved by this.

In a further embodiment variant of the computer-implemented method for providing a blood flow parameter set for a vascular malformation, a vessel section model may be determined in act c2) based on the segmented vascular malformation by adaptation of a volume mesh model. In a further act e3), a porosity parameter may be determined for the vascular malformation based on the vessel section model. In addition, in a further act e4), a permeability parameter may be determined for the vascular malformation based on the vessel section model. Also, in act f1), a pressure ratio between the at least one afferent vessel and the at least one efferent vessel may be determined based on the porosity parameter, the permeability parameter, the average blood flow velocity parameters, and the vessel cross-sectional area parameters.

The volume mesh model may in this case be adapted to the vascular malformation (e.g., to an outer surface of the vascular malformation), such that the volume mesh model extends along the vessel walls of the vascular malformation. In one embodiment, the volume mesh model may be adapted and determined based on the vascular malformation segmented in act c). The volume mesh model may enable a quantitative determination of an outer surface and/or a volume of the vascular malformation.

For example, the volume mesh model may be adapted iteratively to the vascular malformation by minimizing a cost value. The vessel section model may also include information relating to a volume and/or volume portion of a contrast medium within the vascular malformation. For this purpose, the picture elements that have an image value and/or exhibit a change in the image value over time (e.g., the image value and/or the change in the image value over time corresponding to a contrast medium, such as a contrast medium flow) may be segmented in the image of the vessel section. The volume and/or the volume portion of the contrast medium within the vascular malformation may be determined based on the segmented picture elements that correspond to the contrast medium.

The porosity parameter for the vascular malformation may be determined in act e3) based on the volume of the vascular malformation and the volume of the contrast medium within the vascular malformation. The porosity parameter may include information on the capability to absorb a fluid (e.g., blood and/or a contrast medium) within the vascular malformation.

The permeability parameter for the vascular malformation may be determined in act e4) based on the vessel section model (e.g., with reference to a look-up table and/or based on an input by a member of the operating staff) and/or based on at least one parameter (e.g., physiological parameter) of the examination subject (e.g., a blood pressure value and/or body mass and/or age).

In this case, the porosity parameter and the permeability parameter may each describe, for example, a material property of the vascular malformation.

The pressure ratio between the at least one afferent vessel and the at least one efferent vessel may subsequently be determined in act f1) based on the porosity parameter, the permeability parameter, the average blood flow velocity parameters, and the vessel cross-sectional area parameters. In this case, the average blood flow velocity parameters and the vessel cross-sectional area parameters may be taken into account in the determination of the pressure ratio (e.g., as boundary conditions with respect to the blood flow in the vascular malformation, such as at the common cross-sectional areas of the vascular malformation with the at least one afferent or efferent vessel). The determination of the pressure ratio may be based on, for example, Darcy's law. In this case, the pressure ratio may, for example, describe a pressure difference (e.g., a blood pressure difference) between the at least one afferent vessel and the at least one efferent vessel.

Herein, the blood flow parameter set may also include the pressure ratio between the at least one afferent vessel and the at least one efferent vessel. The determination of the pressure ratio may be based, in addition, on a material parameter of the fluid within the vascular malformation (e.g., the contrast medium and/or the blood). The material parameter of the fluid may in this case include, for example, information relating to the dynamic viscosity of the fluid.

In a further embodiment variant of the computer-implemented method for providing a blood flow parameter set for a vascular malformation, act f1) may be performed by applying a trained function to input data. In this case, the input data may be based on the porosity parameter, the permeability parameter, the average blood flow velocity parameters, and the vessel cross-sectional area parameters. At the same time, at least one parameter of the trained function may be based on a comparison between a training pressure ratio and a comparison pressure ratio.

The trained function may be trained by a machine learning method. For example, the trained function may be a neural network (e.g., a convolutional neural network (CNN)) or a network comprising a convolutional layer.

The trained function maps input data to output data. In this case, the output data may also be dependent, for example, on one or more parameters of the trained function. The one or more parameters of the trained function may be determined and/or adjusted by a training process. The determination and/or adjustment of the one or more parameters of the trained function may be based, for example, on a pairing composed of training input data and associated training output data. The trained function is applied to the training input data in order to generate training imaging data. The determination and/or adjustment may be based, for example, on a comparison of the training imaging data with the training output data. Generally, a trainable function (e.g., a function having one or more parameters that have not yet been adjusted) is also referred to as a trained function.

Other terms for trained function are trained imaging rule, imaging rule with trained parameters, function with trained parameters, artificial-intelligence-based algorithm, machine learning algorithm. One example of a trained function is an artificial neural network. The edge weights of the artificial neural network correspond to the parameters of the trained function. The term “neural net” may also be used instead of the term “neural network”. For example, a trained function may also be a deep neural network or deep artificial neural network. A further example of a trained function is a “support vector machine”, and other machine learning algorithms, for example, may also be used as trained functions.

The trained function may be trained, for example, by a backpropagation. First, training imaging data may be determined by applying the trained function to training input data. Next, a deviation between the training imaging data and the training output data may be ascertained by applying an error function to the training imaging data and the training output data. Further, at least one parameter (e.g., a weighting) of the trained function (e.g., of the neural network) may be iteratively adjusted with respect to the at least one parameter of the trained function based on a gradient of the error function. By this, the deviation between the training imaging data and the training output data may be minimized during the training of the trained function.

The trained function (e.g., the neural network) has, for example, an input layer and an output layer. The input layer may in this case be embodied for receiving input data. The output layer may be embodied for providing imaging data. The input layer and/or the output layer may in this case each include a plurality of channels (e.g., neurons).

In one embodiment, at least one parameter of the trained function may be based on a comparison of the training pressure ratio with the comparison pressure ratio. The training pressure ratio and/or the comparison pressure ratio may be determined as part of one or more embodiments of a computer-implemented method for providing a trained function, as will be explained in the further course of the description. For example, the trained function may be provided by an embodiment variant of the computer-implemented method for providing a trained function.

The input data of the trained function may, for example, be based in this case on the porosity parameter, the permeability parameter, the average blood flow velocity parameters, and the vessel cross-sectional area parameters. This enables, for example, all the information contained in the input data in relation to the blood flow dynamics in the vascular malformation to be processed by the trained function.

Further, the input data of the trained function may additionally be based on the vessel section model and/or on the material property of the fluid within the vascular malformation.

In a further embodiment variant of the computer-implemented method for providing a blood flow parameter set for a vascular malformation, a three-dimensional pressure distribution may be determined in addition in act f1).

In this case, the pressure distribution may be determined three-dimensionally (e.g., along the surface of the vascular malformation). The local pressure at the cross-sectional areas of the vascular malformation with the at least one afferent or efferent vessel may be determined as a result. If the vessel section has several afferent or efferent vessels at the vascular malformation, the local pressure associated with each of the afferent or efferent vessels at the respective cross-sectional area with the vascular malformation may be determined.

In a further embodiment variant of the computer-implemented method for providing a blood flow parameter set for a vascular malformation, the image data may map a contrast medium bolus in the vessel section. Act e1) is based on a change in intensity over time in the image of the vessel section due to the contrast medium bolus.

In this case, the contrast medium bolus may describe a temporally and spatially limited contrast medium flow in the vessel section of the examination subject. For example, the contrast medium bolus may at least partially flow through the vessel section of the examination subject during the acquisition of the image data. Herein, a respective status (e.g., a snapshot) of the contrast medium bolus may be mapped in one of the three-dimensional image datasets in each case with associated time information of the time-resolved image of the vessel section. This enables a movement direction (e.g., a flow direction) of the contrast medium bolus to be registered with the aid of the time-resolved image of the vessel section. Further, the metadata of the image data may include at least one parameter (e.g., dynamic information with respect to time) in relation to the contrast medium bolus. Further, a change in intensity over time (e.g., a change in the image values over time) of mutually corresponding picture elements of the multiple three-dimensional image datasets of the time-resolved image of the vessel section may be detected by the temporally and spatially limited contrast medium flow of the contrast medium bolus in the vessel section.

Further, the average blood flow velocity parameter may be determined in act e1) for the at least one afferent vessel and the at least one efferent vessel based on the spatial distance traveled by the contrast medium bolus in the vessel section in a specific time period.

In this case, the spatial distance traveled by the contrast medium bolus in the vessel section may, for example, be determined three-dimensionally by a threshold value with respect to the image values of the picture elements and the respectively associated time information of the three-dimensional image datasets. An average blood flow velocity parameter may, for example, be determined in each case as the quotient from the spatial distance traveled by the contrast medium bolus in the respective afferent or efferent vessel and the length of time required therefor.

This enables a particularly accurate determination of the average blood flow velocity parameter for the at least one afferent vessel and the at least one efferent vessel.

In a further embodiment variant of the computer-implemented method for providing a blood flow parameter set for a vascular malformation, the porosity parameter may be determined in act e3) based on a ratio between a volume of the vascular malformation and a volume of the contrast medium bolus within the vascular malformation.

In this case, the porosity parameter may be determined as a ratio (e.g., a quotient) between the volume of the contrast medium within the vascular malformation and the volume of the vascular malformation. For this purpose, the volume of the vascular malformation may be ascertained by the vessel section model (e.g., the volume mesh model). Further, the picture elements of the time-resolved image of the vessel section that have an image value and/or exhibit a change over time in the image value (e.g., a change in intensity, the image value, and/or the change over time in the image value corresponding to a contrast medium, such as a contrast medium flow and/or contrast medium bolus) may be segmented in the image of the vessel section. The volume and/or the volume portion of the contrast medium within the vascular malformation may be determined based on the segmented picture elements that correspond to the contrast medium and/or contrast medium bolus.

A particularly accurate determination of the porosity parameter may be made possible as a result.

In a further embodiment variant of the computer-implemented method for providing a blood flow parameter set for a vascular malformation, the image of the vessel section may have a number of voxels. In this case, the reconstruction in act b) may assign a bolus arrival time to the voxels in which the at least one afferent vessel and/or the at least one efferent vessel and/or the vascular malformation is imaged.

In this case, each of the multiple three-dimensional image datasets of the time-resolved image of the vessel section may include the multiple voxels. For example, the three-dimensional image datasets may each include multiple voxels. The voxels of the multiple three-dimensional image datasets that image the same part of the vessel section at different acquisition time points correspond to one another. In one embodiment, the bolus arrival time may describe a time point (e.g., a relative time point) at which a predefined threshold value with respect to the image value of a voxel is exceeded.

For this purpose, the time-resolved image of the vessel section may, for example, include a time intensity curve for each voxel, where the bolus arrival time may be determined according to the time based on the time information when the predefined threshold value is exceeded (e.g., for the first time) and/or by determination of the first derivation and/or the second derivation of the time intensity curves. In this case, the determination of the bolus arrival time may, for example, be limited to the voxels of the image of the vessel section that image the at least one afferent vessel and/or the at least one efferent vessel and/or the vascular malformation. The bolus arrival time may, for example, be determined relative to the acquisition time point of the first image data of the vessel section.

In a further embodiment variant of the computer-implemented method for providing a blood flow parameter set for a vascular malformation, act d1) and/or act d2) may be based on a comparison of the bolus arrival time of different voxels of the image of the vessel section.

In this case, the at least one afferent vessel may be identified in act d1), in that the at least one afferent vessel has a shorter bolus arrival time compared to the at least one efferent vessel. For example, the at least one afferent vessel and/or the at least one efferent vessel may be identified by a comparison of the bolus arrival times of different voxels that correspond to an image of the respective vessel.

Further, the average blood flow velocity parameter of the at least one afferent vessel and of the at least one efferent vessel may be determined based on the respective bolus arrival times of the voxels along the respective vessels.

In a further embodiment variant of the computer-implemented method for providing a blood flow parameter set for a vascular malformation, the blood flow parameter set may include a temporal blood volume flow parameter in each case for the at least one afferent vessel and the at least one efferent vessel. In this case, the temporal blood flow parameters may be determined based on the respective average blood flow velocity parameter and the respective vessel cross-sectional area parameter.

Also, the blood flow parameter set may include one blood flow parameter in each case for the at least one afferent vessel and the at least one efferent vessel. Further, the temporal blood volume flow parameters include, for example, information in each case about the volume flow rate (volume flow ratio (VFR)) relating to the at least one afferent vessel and the at least one efferent vessel. One of the temporal blood flow parameters may be determined in each case based on the average blood flow velocity parameter and the vessel cross-sectional area parameter of the respective afferent or efferent vessel (e.g., as a product and/or sum).

This enables a particularly accurate characterization of the at least one afferent vessel and of the at least one efferent vessel in terms of the temporal blood volume flow.

The present embodiments relate, in a second aspect, to a computer-implemented method for providing a trained function. In one embodiment, in a first act, average training blood flow velocity parameters, training vessel cross-sectional area parameters, and a segmented training vascular malformation are received by applying an embodiment variant of the proposed computer-implemented method for providing a blood flow parameter set for a vascular malformation. In this case, the average blood flow velocity parameters are provided as the average training blood flow velocity parameters, the vessel cross-sectional area parameters are provided as the training vessel cross-sectional area parameters, and the segmented vascular malformation are provided as the training vascular malformation. In a second act, a training vessel section model is determined based on the training vascular malformation by adapting a volume mesh model. In a third act, a training porosity parameter is determined for the training vascular malformation based on the training vessel section model. At the same time, a training permeability parameter is also determined for the training vascular malformation based on the training vessel section model. In a fourth act, a comparison pressure ratio between the at least one afferent vessel and the at least one efferent vessel is determined based on the training porosity parameter, the training permeability parameter, the average training blood flow velocity parameters, and the training vessel cross-sectional area parameters. Further, in a fifth act, a training pressure ratio between the at least one afferent vessel and the at least one efferent vessel is determined by applying the trained function to input data. The input data is based on, for example, the training porosity parameter, the training permeability parameter, the average training blood flow velocity parameters, and the training vessel cross-sectional area parameters. Next, in a sixth act, at least one parameter of the trained function is adjusted based on a comparison between the training pressure ratio and the comparison pressure ratio. The trained function is provided in a seventh act. The order of the above-described acts in this process may, for example, be variable.

The receiving of the average training blood flow velocity parameters, the training vessel cross-sectional area parameters, and/or the training vascular malformation may, for example, include an acquisition and/or a readout of a computer-readable data storage medium and/or a receiving from a computer-readable data storage medium and/or a receiving from a data storage (e.g., a database). Further, the average training blood flow velocity parameters, the training vessel cross-sectional area parameters, and/or the training vascular malformation may be provided by a provider unit of a medical imaging device.

The average training blood flow velocity parameters may, for example, include all properties of the blood flow velocity parameters that have been described in relation to the computer-implemented method for providing a blood flow parameter set for a vascular malformation, and vice versa. Further, the training vessel cross-sectional area parameters may include all properties of the vessel cross-sectional area parameters that have been described in relation to the computer-implemented method for providing a blood flow parameter set for a vascular malformation, and vice versa. Analogously thereto, the training vascular malformation may include all properties of the segmented vascular malformation that have been described in relation to the computer-implemented method for providing a blood flow parameter set for a vascular malformation, and vice versa. For example, the average training blood flow velocity parameters may be average blood flow velocity parameters, and/or the training vessel cross-sectional area parameters may be vessel cross-sectional area parameters, and/or the training vascular malformation may be a segmented vascular malformation. In addition, the average training blood flow velocity parameters, the training vessel cross-sectional area parameters, and/or the training vascular malformation may be simulated.

The training vessel section model may, for example, be determined based on the training vascular malformation analogously to the vessel section model according to act c2) of the proposed computer-implemented method for providing a blood flow parameter set for a vascular malformation. Further, the training porosity parameter and the training permeability parameter may be determined in each case based on the training vessel section model analogously to acts e3) and e4) of the computer-implemented method of one or more of the present embodiments for providing a blood flow parameter set for a vascular malformation.

The comparison pressure ratio between the at least one afferent vessel and the at least one efferent vessel may, for example, be determined based on the training porosity parameter, the training permeability parameter, the average training blood flow velocity parameters, and the training vessel cross-sectional area parameters. The average training blood flow velocity parameters and the training vessel cross-sectional area parameters may be taken into account in this case in the determination of the comparison pressure ratio, for example, as boundary conditions with respect to the blood flow in the training vascular malformation (e.g., at the common cross-sectional areas of the training vascular malformation with the at least one afferent or efferent vessel). For example, the training pressure ratio may be determined based on Darcy's law. In this case, the training pressure ratio may describe a pressure difference (e.g., a blood pressure difference) between the at least one afferent vessel and the at least one efferent vessel.

Further, a training pressure ratio may be determined by applying the trained function to input data. The input data is based on the training porosity parameter, the training permeability parameters, the average training blood flow velocity parameters, and the training vessel cross-sectional area parameters. Further, at least one parameter of the trained function may be adjusted by a comparison (e.g., a cost value, such as a normalized difference and/or a scalar product) between the training pressure ratio and the comparison pressure ratio.

This enables, for example, an accuracy in the determination of the blood flow parameter set (e.g., of the pressure ratio) for the vascular malformation to be improved by applying the trained function to the input data.

The providing of the trained function may, for example, include a storing on a computer-readable storage medium and/or a transfer to a provider unit.

According to a further embodiment variant of the computer-implemented method for providing a trained function, a three-dimensional comparison pressure distribution (e.g., along the surface of the training vascular malformation) may be determined based on the training porosity parameter, the training permeability parameter, the average training blood flow velocity parameters, and the training vessel cross-sectional area parameters. The three-dimensional comparison pressure distribution may also be determined based on, for example, the training vessel section model. Further, a three-dimensional training pressure distribution may be determined by applying the trained function to the input data, in which case at least one parameter of the trained function may be adjusted based on a comparison (e.g., a voxel-by-voxel comparison) between the comparison pressure distribution and the training pressure distribution. The input data of the trained function may also be based on, for example, the training vessel section model.

In one embodiment, the method of one or more of the present embodiments may be employed to provide a trained function that may be used in an embodiment variant of the computer-implemented method for providing a blood flow parameter set for a vascular malformation.

The present embodiments relate, in a third aspect, to a provider unit including a computing unit and an interface. In this case, the interface may be embodied for receiving time-resolved image data. The computing unit may be embodied for reconstructing a time-resolved image of the vessel section from the image data. Further, the computing unit may be embodied for segmenting the vascular malformation in the image of the vessel section. The computing unit may also be embodied for identifying at least one afferent vessel at the vascular malformation based on the image of the vessel section. Further, the computing unit may be embodied for identifying at least one efferent vessel at the vascular malformation based on the image of the vessel section. The computing unit may also be embodied for determining an average blood flow velocity parameter in each case for the at least one afferent vessel and the at least one efferent vessel. Further, the computing unit may be embodied for determining a vessel cross-sectional area parameter in each case for the at least one afferent vessel and the at least one efferent vessel. In addition, the computing unit may be embodied for determining the blood flow parameter set for the vascular malformation based on the average blood flow velocity parameters and the vessel cross-sectional area parameters. The interface may further be embodied for providing the blood flow parameter set for the vascular malformation.

Such a provider unit may be embodied for carrying out the above-described inventive methods for providing a blood flow parameter set for a vascular malformation and corresponding aspects. The provider unit is embodied for carrying out the methods and the corresponding aspects in that the interface and the computing unit are embodied for performing the corresponding method acts.

The advantages of the proposed provider unit substantially correspond to the advantages of the proposed computer-implemented method for providing a blood flow parameter set for a vascular malformation. Features, advantages, or alternative embodiment variants mentioned in this regard may also be applied equally to the other subject matters, and vice versa.

The present embodiments relate, in a fourth aspect, to a training unit that is embodied for carrying out the above-described computer-implemented methods for providing a trained function and corresponding aspects. The training unit includes, for example, a training interface and a training computing unit. The training unit is embodied to carry out the methods and corresponding aspects in that the training interface and the training computing unit are embodied to perform the corresponding method acts.

In this case, the training interface may be embodied for receiving average training blood flow velocity parameters, training vessel cross-sectional area parameters, and a training vascular malformation by applying an embodiment variant of the computer-implemented method for providing a blood flow parameter set for a vascular malformation. The average blood flow velocity parameters may be provided as, for example, the average training blood flow velocity parameters, the vessel cross-sectional area parameters may be provided as, for example, the training vessel cross-sectional area parameters, and the segmented vascular malformation may be provided as, for example, as the training vascular malformation. The training computing unit may be embodied for determining a training vessel section model based on the training vascular malformation by adapting a volume mesh model. Further, the training computing unit may be embodied for determining a training porosity parameter for the training vascular malformation based on the training vessel section model. In addition, the training computing unit may be embodied for determining a training permeability parameter for the training vascular malformation based on the training vessel section model. Further, the training computing unit may be embodied for determining a comparison pressure ratio between the at least one afferent vessel and the at least one efferent vessel based on the training porosity parameter, the training permeability parameter, the average training blood flow velocity parameters, and the training vessel cross-sectional area parameters. Further, the training computing unit may be embodied for determining a training pressure ratio between the at least one afferent vessel and the at least one efferent vessel by applying the trained function to input data. The input data is based on the training porosity parameter, the training permeability parameter, the average training blood flow velocity parameters, and the training vessel cross-sectional area parameters. The training computing unit may also be embodied for adjusting at least one parameter of the trained function based on a comparison between the training pressure ratio and the comparison pressure ratio. Further, the training interface may be embodied for providing the trained function.

The advantages of the proposed training unit substantially correspond to the advantages of the proposed computer-implemented method for providing a trained function. Features, advantages, or alternative embodiment variants mentioned in this regard may also be applied equally to the other subject matters, and vice versa.

The present embodiments relate, in a fifth aspect, to a medical imaging device including a provider unit. The medical imaging device (e.g., the provider unit) is in this case embodied for carrying out a computer-implemented method of one or more of the present embodiments for providing a blood flow parameter set for a vascular malformation. For example, the medical imaging device may be embodied as a medical X-ray apparatus (e.g., a C-arm X-ray apparatus) and/or as a computed tomography system (CT) and/or as a magnetic resonance system (MRT) and/or as a sonography apparatus and/or as a positron emission tomography system (PET). At the same time, the medical imaging device may also be embodied for acquiring and/or for receiving and/or for providing the time-resolved image data.

The advantages of the medical imaging device substantially correspond to the advantages of the computer-implemented methods for providing a blood flow parameter set for a vascular malformation. Features, advantages, or alternative embodiment variants mentioned in this regard may also be applied equally to the other subject matters, and vice versa.

The present embodiments relate, in a sixth aspect, to a computer program product including a computer program that may be loaded directly into a memory of a provider unit, having program sections for performing all the acts of the computer-implemented method for providing a blood flow parameter set for a vascular malformation and/or a corresponding aspect when the program sections are executed by the provider unit. Alternatively or additionally, the computer program may be loaded directly into a training memory of a training unit, having program sections for performing all the acts of a method of one or more of the present embodiments for providing a trained function and/or a corresponding aspect when the program sections are executed by the training unit.

The present embodiments relate, in a seventh aspect, to a computer-readable storage medium on which program sections that are readable and executable by a provider unit (e.g., including one or more processors) are stored in order to perform all the acts of the computer-implemented method for providing a blood flow parameter set for a vascular malformation and/or a corresponding aspect when the program sections are executed by the provider unit. Alternatively or additionally, the program sections are readable and executable by a training unit, and are stored in order to perform all the acts of the method for providing a trained function and/or a corresponding aspect when the program sections are executed by the training unit.

The present embodiments relate, in an eighth aspect, to a computer program or computer-readable storage medium including a trained function provided by a computer-implemented method of one or more of the present embodiments or a corresponding aspect.

An implementation to a large extent in the form of software has the advantage that provider units and/or training units already used previously in the prior art may also be easily upgraded by a software update in order to operate in the manner according to the present embodiments. In addition to the computer program, such a computer program product may, where appropriate, include additional constituent parts such as, for example, a set of documentation and/or additional components, as well as hardware components, such as, for example, hardware keys (e.g., dongles, etc.) to enable use of the software.

### BRIEF DESCRIPTION OF THE DRAWINGS

Exemplary embodiments of the invention are shown in the drawings and are described in more detail hereinbelow. The same reference signs are used for like features in different figures, in which:

FIG. 1 shows a schematic view of an embodiment of a computer-implemented method for providing a blood flow parameter set for a vascular malformation;

FIG. 2 shows a schematic view of one example of data flow in the computer-implemented method for providing a blood flow parameter set for a vascular malformation;

FIGS. 3 to 6 show schematic views of different embodiments of the computer-implemented method for providing a blood flow parameter set for a vascular malformation;

FIG. 7 shows a schematic view of one embodiment of a computer-implemented method for providing a trained function;

FIG. 8 shows a schematic view of one embodiment of a provider unit;

FIG. 9 shows a schematic view of one embodiment of a training unit; and

FIG. 10 shows a schematic view of one embodiment of a medical C-arm X-ray apparatus.

### DETAILED DESCRIPTION

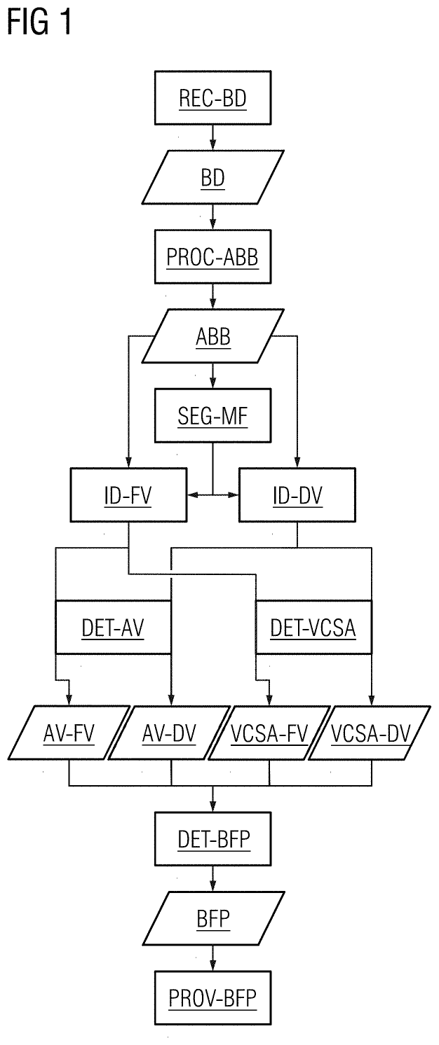

FIG. 1 schematically illustrates an embodiment of a computer-implemented method for providing a blood flow parameter set for a vascular malformation. In the embodiment shown, in a first act a), time-resolved image data BD (e.g., image data) may be received REC-BD. The image data BD maps a change over time in a vessel section VS of an examination subject 31. Further, the vessel section VS may include the vascular malformation MF. In a second act b), a time-resolved image ABB of the vessel section VS may be reconstructed PROC-ABB from the image data BD. After this, in a third act c), the vascular malformation MF may be segmented SEG-MF in the image ABB of the vessel section VS. Further, in act d1), at least one afferent vessel FV may be identified ID-FV at the vascular malformation MF based on the image ABB of the vessel section VS. Also, in a further act d2), at least one efferent vessel DV may be identified ID-DV at the vascular malformation MF based on the image ABB of the vessel section VS. After this, in act e1), an average blood flow velocity parameter may be determined DET-AV in each case for the at least one afferent vessel AV-FV and the at least one efferent vessel AV-DV. Further, in a further act e2), a vessel cross-sectional area parameter may be determined DET-VCSA in each case for the at least one afferent vessel VCSA-FV and the at least one efferent vessel VCSA-DV. After this, in act f1), the blood flow parameter set BFP for the vascular malformation MF may be determined DET-BFP based on the average blood flow velocity parameters AV-FV, VA-DV and the vessel cross-sectional area parameters VCSA-FV, VCSA-DV.

The blood flow parameter set BFP may, for example, include information concerning the volume flow rate in relation to the at least one afferent vessel and/or the at least one efferent vessel. The volume flow rate \dot{V} may in this case be determined, for example, as a product from the respective average blood flow velocity parameter AV-FV or AV-DV and the associated vessel cross-sectional area parameter VCSA-FV or VCSA-DV:

\dot{V}_{FV}=AV-FV·VCSA-FV (1)

\dot{V}_{DV}=AV-DV·VCSA-DV (2)

The blood flow parameter set BFP may further be provided PROV-BFP in act g).

In addition, the image data BD may map a contrast medium bolus in the vessel section VS. In such a case, act e1) may be based on a change in intensity over time in the image ABB of the vessel section VS due to the contrast medium bolus.

The image ABB of the vessel section VS may also have a number of voxels. The reconstruction PROC-ABB in act b) assigns a bolus arrival time to each of the voxels in which the at least one afferent vessel FV and/or the at least one efferent vessel DV and/or the vascular malformation MF is depicted. In this case, the identification of the at least one afferent vessel ID-FV in act d1) and/or the identification of the at least one efferent vessel ID-DV in act d2) may be based on a comparison of the bolus arrival time of different voxels of the image ABB of the vessel section VS.

The blood flow parameter set BFP may also include a temporal blood volume flow parameter in each case for the at least one afferent vessel FV and the at least one efferent vessel DV. In this case, the temporal blood volume flow parameter may be determined based on the respective average blood flow velocity parameter AV-FV or AV-DV and the respective vessel cross-sectional area parameter VCSA-FV or VCSA-DV.

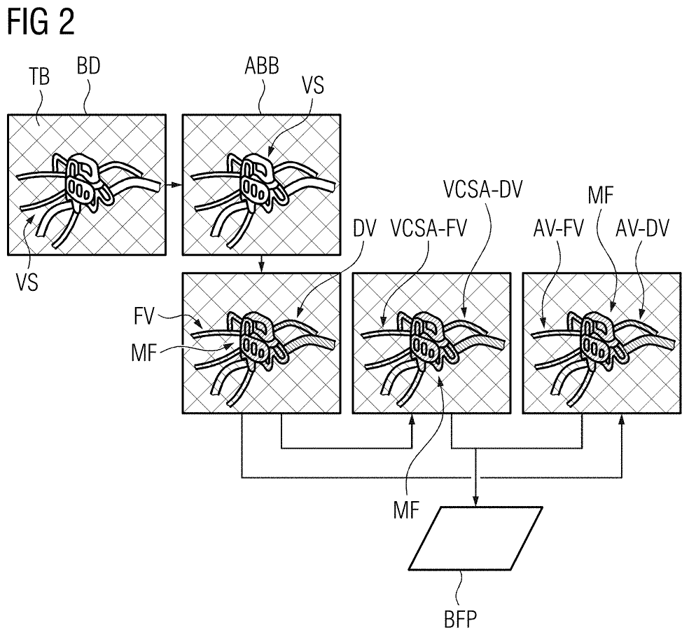

FIG. 2 schematically illustrates the data flow of an embodiment variant of the method for providing PROV-BFP a blood flow parameter set BFP for a vascular malformation MF. The vessel section VS of the examination subject 31 is depicted in the image data BD against the tissue background TB. Further, the vessel section VS in the image ABB of the vessel section VS may be reconstructed three-dimensionally. In this case, the image ABB of the vessel section VS may include multiple three-dimensional image datasets to each of which time information is assigned. This enables the image ABB of the vessel section also to map a change over time in the vessel section VS three-dimensionally. After the segmenting SEG-MF of the vascular malformation in the image ABB of the vessel section VS, the at least one afferent vessel FV and the at least one efferent vessel DV at the vascular malformation MF may be identified ID-FV, ID-DV. Further, the vessel cross-sectional area parameters may be determined DET-VCSA for the at least one afferent vessel VCSA-FV and the at least one efferent vessel VCSA-DV. In addition, the average blood flow velocity parameters may be determined DET-AV for the at least one afferent vessel AV-FV and the at least one efferent vessel AV-DV. After this, the blood flow parameter set BFP for the vascular malformation MF may be determined DET-BFP based on the average blood flow velocity parameters AV-FV or AV-DV and the vessel cross-sectional area parameters VCSA-FV or VCSA-DV.

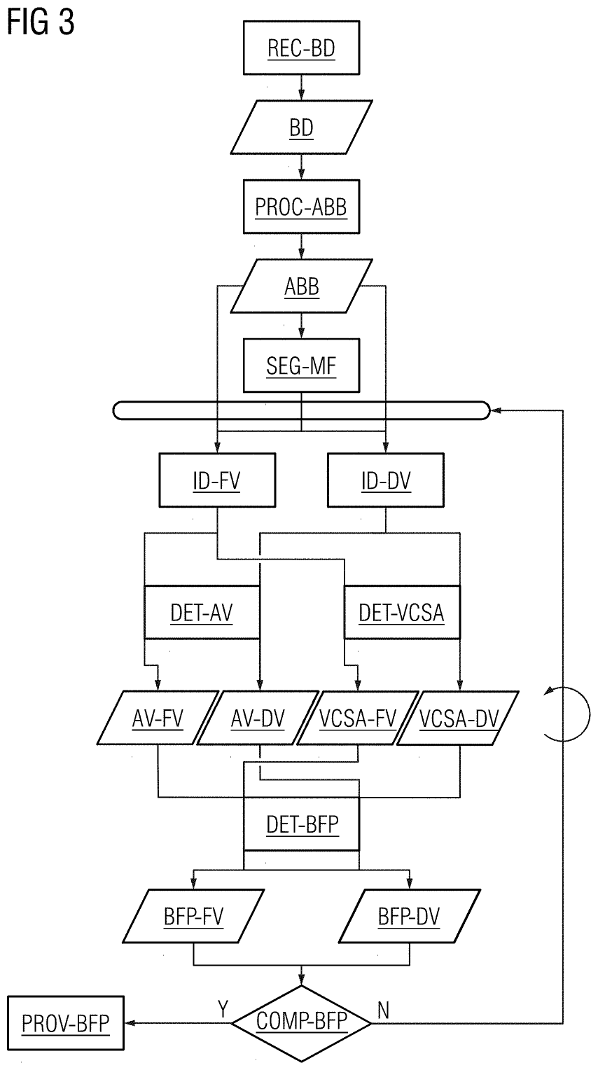

In the embodiment of the computer-implemented method for providing PROV-BFP a blood flow parameter set BFP for a vascular malformation MF illustrated schematically in FIG. 3, the blood flow parameter set BFP may include at least one first blood flow parameter BFP-FV corresponding to the at least one afferent vessel FV. The blood flow parameter set BFP may also include at least one second blood flow parameter BFP-DV corresponding to the at least one efferent vessel DV. In this case, the method may also include act f2), in which a sum of the at least one first blood flow parameter BFP-FV is compared COMP-BFP with a sum of the at least one second blood flow parameter BFP-DV. The comparison may include, for example, a validity condition with regard to the sum of the volume flow rate of the at least one afferent vessel and the sum of the volume flow rate of the at least one efferent vessel:

Σ\dot{V}_{FV}=Σ\dot{V}_{DV} (3)

The method may in this case be executed repeatedly starting at act d1) as of a predetermined discrepancy between the sums. If the result of the comparison is that the sums lie within the predetermined discrepancy, the blood flow parameter set BFP may be provided PROV-BFP.

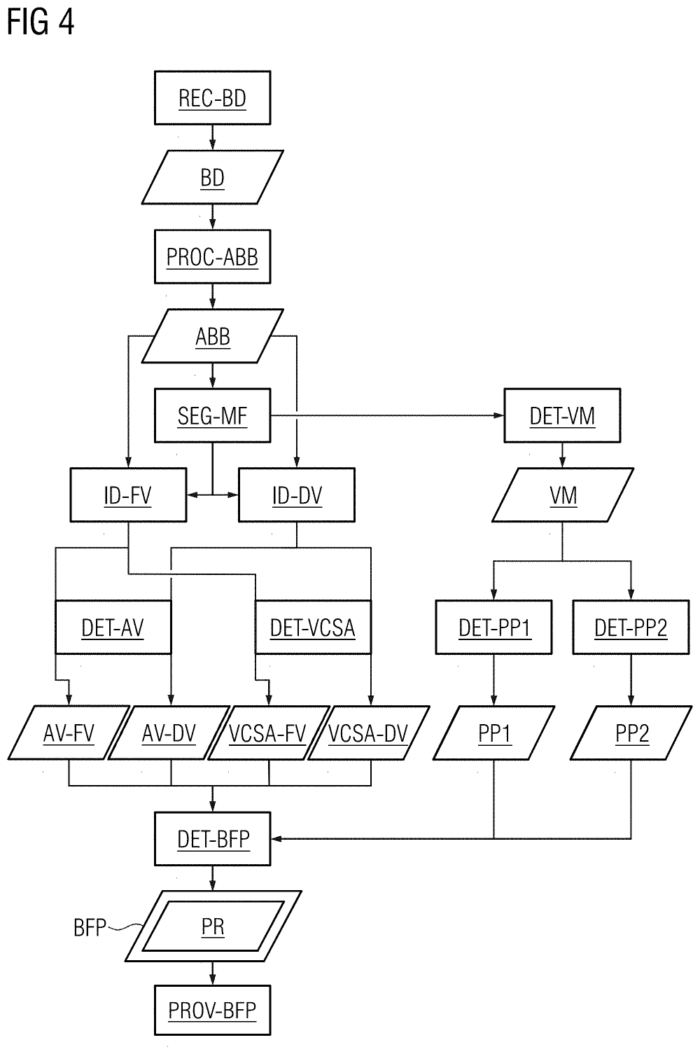

FIG. 4 schematically illustrates a further embodiment of the computer-implemented method for providing PROV-BFP a blood flow parameter set BFP for a vascular malformation MF. In this case, in act c2), a vessel section model VM may be determined DET-VM based on the segmented vascular malformation MF by adapting a volume mesh model. Next, a porosity parameter PP1 may be determined DET-PP1 for the vascular malformation MF based on the vessel section model VM. A permeability parameter PP2 may also be determined DET-PP2 for the vascular malformation MF based on the vessel section model VM. In addition, in act f1), a pressure ratio PR between the at least one afferent vessel FV and the at least one efferent vessel DV may be determined DET-BFP based on the porosity parameter PP1, the permeability parameter PP2, the average blood flow velocity parameters AV-FV and AV-DV, and the vessel cross-sectional area parameters VCSA-FV and VCSA-DV. Further, a three-dimensional pressure distribution may be determined in act f1).

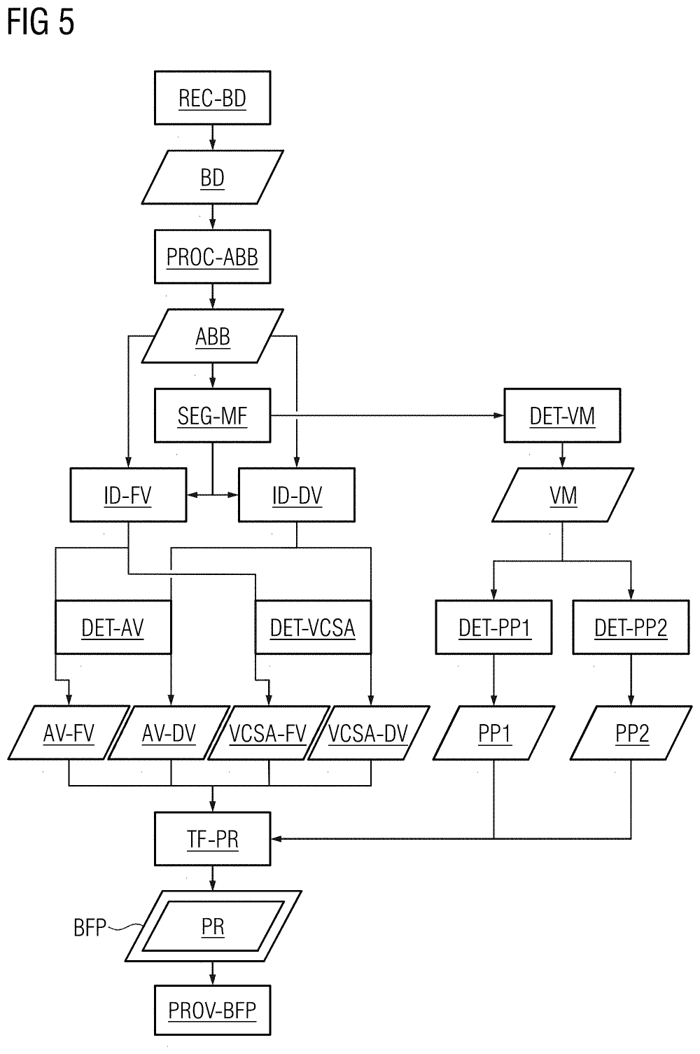

In the embodiment of the computer-implemented method for providing PROV-BFP a blood flow parameter set BFP for a vascular malformation MF schematically illustrated in FIG. 5, act f1) may be performed by applying a trained function TF-PR to input data. The input data may be based here on the porosity parameter PP1, the permeability parameter PP2, the average blood flow velocity parameters AV-FV or AV-DV, and the vessel cross-sectional area parameters VCSA-FV or VCSA-DV.

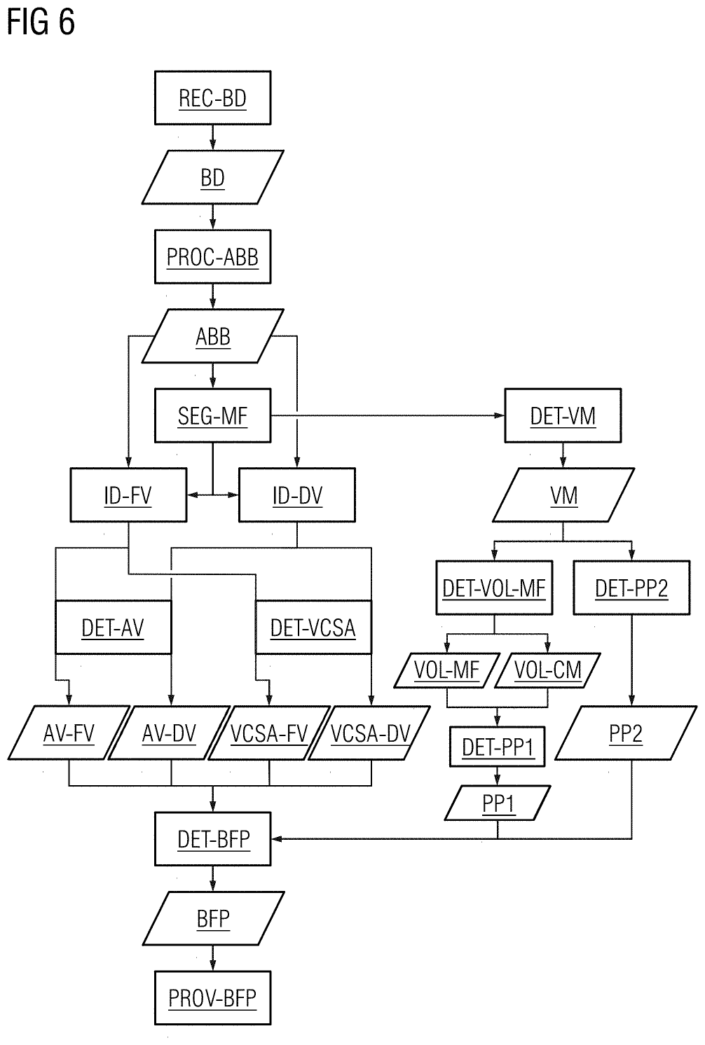

FIG. 6 schematically illustrates a further embodiment of the computer-implemented method for providing PROV-BFP a blood flow parameter set BFP for a vascular malformation MF. In this case, in act e3), a spatial volume VOL-MF of the vascular malformation MF may, for example, be determined DET-VOL-MF based on the vessel section model VM. A spatial volume VOL-CM of the contrast medium bolus within the vascular malformation MF may also be determined DET-VOL-CM in act e3). The porosity parameter PP1 may then be determined DET-PP1 based on a ratio between the volume of the vascular malformation VOL-MF and the volume of the contrast medium bolus VOL-CM within the vascular malformation MF.

Darcy's law may be applied for the flow Q of a fluid in a porous medium (e.g., the vascular malformation MF). This is derivable by a homogenization of the Navier-Stokes equations:

$$\dot{V} = \frac{PP 2 \cdot CSA \cdot ( {p}_{1} - {p}_{2} )}{μ \cdot L} , \qquad ( 4 )$$

where PP2 denotes the permeability parameter of the vascular malformation, μ denotes the dynamic viscosity of the fluid, CSA denotes the vessel cross-sectional area (e.g., at the cross-sectional areas with the at least one afferent and efferent vessel FV and DV respectively), and L denotes a spatial distance between two spatial points, with the pressure p₁ and p₂ prevailing respectively at the two spatial points.

From Equation (4), it may be derived that:

$$q = \frac{\dot{V}}{CSA} = \frac{PP 2}{μ} \cdot \text{∇} p , \qquad ( 5 )$$

where ∇p denotes the pressure gradient between the cross-sectional areas of the vascular malformation MF with the at least one afferent and efferent vessel FV and DV, respectively (e.g., along the spatial distance L), and q denotes the volume flow rate normalized to the vessel cross-sectional area CSA.

It follows from this that the pressure gradient ∇p is indirectly proportional to the permeability parameter PP2 of the vessel:

$$\text{∇} p ∝ \frac{1}{PP 2} . \qquad ( 6 )$$

The permeability parameter PP2 may be predefinable at the same time. Further, the porosity parameter PP1 for the vascular malformation MF may be determined as:

$$PP 1 = \frac{{VOL}_{V}}{VOL \text{-} MF} , \qquad ( 7 )$$

where VOL_{V} denotes the spatial volume of the vascular malformation MF that may not be filled by a fluid, where:

VOL_{V}=VOL-MF−VOL-CM (8).

Further, an average velocity ν of the fluid may be determined as:

$$v = \frac{q}{PP 1} . \qquad ( 9 )$$

Darcy's law may be applied, for example, for a laminar flow that often occurs in hemodynamics. Alternatively, Equation (4) may be supplemented by an inertia term (e.g., a Forchheimer term).

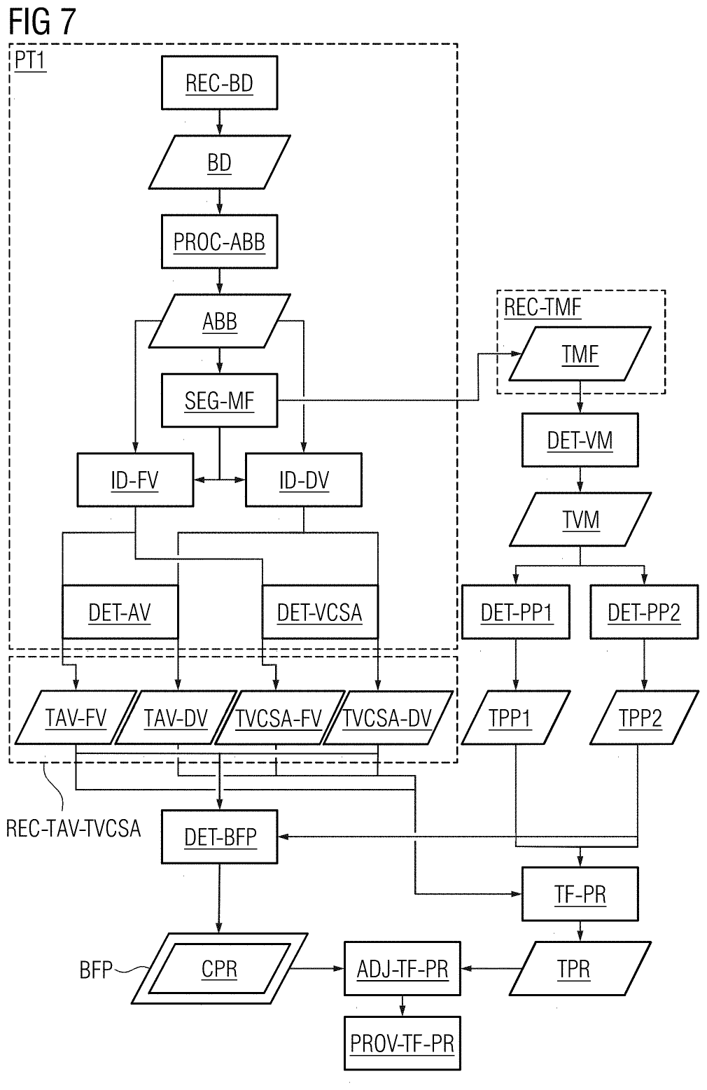

FIG. 7 schematically illustrates an embodiment of the computer-implemented method for providing PROV-TF-PR a trained function TF-PR. In this case, average training blood flow velocity parameters TAV-FV and TAV-DV and training vessel cross-sectional area parameters TVCSA-FV and TVCSA-DV may be received REC-TAV-TVCSA by applying PT1 an embodiment of the computer-implemented method for providing a blood flow parameter set PROV-BFP for a vascular malformation MF. At the same time, the average blood flow velocity parameters AV-FV and AV-DV may be provided as the training blood flow velocity parameters TAV-FV and TAV-DV. Further, the vessel cross-sectional area parameters VCSA-FV and VCSA-DV may be provided as the training vessel cross-sectional area parameters TVCSA-FV and TVCSA-DV. Also, a training vascular malformation TMF may be received REC-TMF. The segmented vascular malformation MF is provided as the training vascular malformation TMF. A training vessel section model TVM may be determined DET-VM based on, for example, the training vascular malformation TMF by adapting a volume mesh model. A training porosity parameter TPP1 may also be determined DET-PP1 for the training vascular malformation TMF based on the training vessel section model TVM. In addition, a training permeability parameter TPP2 may be determined DET-PP2 for the training vascular malformation TMF based on the training vessel section model TVM. After this, a comparison pressure ratio CPR between the at least one afferent vessel FV and the at least one efferent vessel DV may be determined DET-BFP based on the training porosity parameter TPP1, the training permeability parameter TPP2, the average blood flow velocity parameters TAV-FV and TAV-DV, and the training vessel cross-sectional area parameters TVCSA-FV and TVCSA-DV. In a further act, a training pressure ratio TPR between the at least one afferent vessel FV and the at least one efferent vessel DV may be determined by applying the trained function TF-PR to input data. In the process, the input data may be based on the training porosity parameter TPP1, the training permeability parameter TPP2, the average training blood flow velocity parameters TAV-FV and TAV-DV, and the training vessel cross-sectional area parameters TVCSA-FV and TVCSA-DV. Next, at least one parameter of the trained function TF-PR may be adjusted ADJ-TF-PR based on a comparison between the training pressure ratio TPR and the comparison pressure ratio CPR. The trained function TF-PR may be provided PROV-TF-PR in a further act.

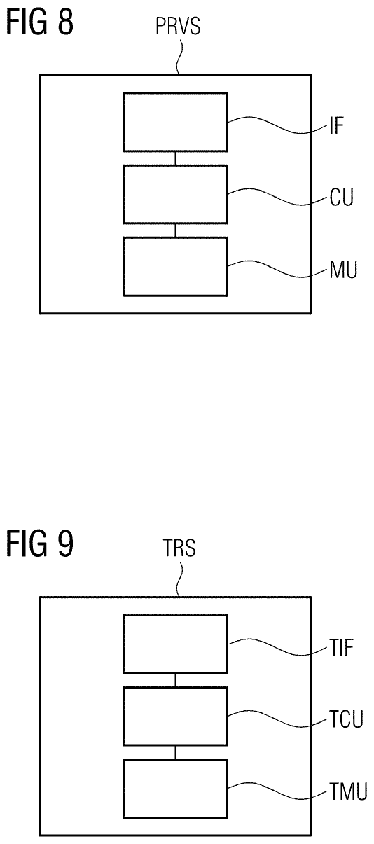

FIG. 8 schematically illustrates one embodiment of a provider unit PRVS including an interface IF, a computing unit CU, and a memory unit MU. The provider unit PRVS may be embodied to carry out a computer-implemented method of one or more of the present embodiments for providing PROV-BFP a blood flow parameter set BFP for a vascular malformation MF and corresponding aspects, in that the interface IF and the computing unit CU are embodied to perform the corresponding method acts. The interface IF may be embodied in this case for receiving REC-BD the time-resolved image data BD. Further, the computing unit CU may be embodied to reconstruct PROC-ABB the time-resolved image ABB of the vessel section VS from the image data BD. The computing unit CU may be further embodied to segment SEG-MF the vascular malformation MF in the image ABB of the vessel section VS. The computing unit CU may also be embodied to identify ID-FV at least one afferent vessel FV at the vascular malformation MF based on the image ABB of the vessel section VS. The computing unit CU may further be embodied to identify ID-DV at least one efferent vessel DV at the vascular malformation MF based on the image ABB of the vessel section VS. The computing unit CU may further be embodied to determine DET-AV an average blood flow velocity parameter in each case for the at least one afferent vessel AV-FV and the at least one efferent vessel AV-FV. Further, the computing unit CU may be embodied for determining DET-VCSA a vessel cross-sectional area parameter in each case for the at least one afferent vessel VCSA-FV and the at least one efferent vessel VCSA-DV. The computing unit CU may also be embodied for determining DET-BFP the blood flow parameter set BFP for the vascular malformation MF based on the average blood flow velocity parameters AV-FV and AV-DV and the vessel cross-sectional area parameters VCSA-FV and VCSA-DV. In addition, the interface IF may be embodied for providing PROV-BFP the blood flow parameter set BFP for the vascular malformation MF.

FIG. 9 schematically illustrates one embodiment of a training unit TRS including a training interface TIF, a training computing unit TCU, and a training memory unit TMU. The training unit TRS may be embodied to carry out an embodiment of a computer-implemented method for providing a trained function PROV-TF-PR and corresponding aspects, in that the training interface TIF and the training computing unit TCU are embodied to perform the corresponding method acts.

In this case, the training interface TIF may be embodied for receiving the average training blood flow velocity parameters TAV-FV and TAV-DV, the training vessel cross-sectional area parameters TVCSA-FV and TVCSA-DV, and the training vascular malformation TMF by applying a variant of the computer-implemented method for providing PROV-BFP a blood flow parameter set BFP for a vascular malformation MF. In this case, the average blood flow velocity parameters AV-FV and AV-DV may be provided as the average training blood flow velocity parameters TAV-FV and TAV-DV, the vessel cross-sectional area parameters VCSA-FV and VCSA-DV may be provided as the training vessel cross-sectional area parameters TVCSA-FV and TVCSA-DV, and the segmented vascular malformation MF may be provided as the training vascular malformation TMF. Further, the training computing unit TCU may be embodied for determining DET-VM a training vessel section model TVM based on the training vascular malformation TMF by adapting a volume mesh model. Further, the training computing unit TCU may be embodied for determining DET-PP1 a training porosity parameter TPP1 for the training vascular malformation TMF based on the training vessel section model TVM. Further, the training computing unit TCU may be embodied for determining DET-PP2 a training permeability parameter TPP2 for the training vascular malformation TMF based on the training vessel section model TVM. Further, the training computing unit TCU may be embodied for determining DET-BFP a comparison pressure ratio CPR between the at least one afferent vessel FV and the at least one efferent vessel DV based on the training porosity parameter TPP1, the training permeability parameter TPP2, the average training blood flow velocity parameters TAV-FV and TAV-DV, and the training vessel cross-sectional area parameters TVCSA-FV and TVCSA-DV. Further, the training computing unit TCU may be embodied for determining a training pressure ratio TPR between the at least one afferent vessel FV and the at least one efferent vessel DV by applying the trained function TF-PR to input data. The input data is based on the training porosity parameter TPP1, the training permeability parameter TPP2, the average training blood flow velocity parameters TAV-FV and TAV-DV, and the training vessel cross-sectional area parameters TVCSA-FV and TVCSA-DV. Further, the training computing unit TCU may be embodied for adjusting ADJ-TF-PR at least one parameter of the trained function TF-PR based on a comparison between the training pressure ratio TPR and the comparison pressure ratio CPR. Further, the training interface TCU may be embodied for providing PROV-TF-PR the trained function TF-PR.

The provider unit PRVS and/or the training unit TRS may, for example, be a computer, a microcontroller, or an integrated circuit. Alternatively, the provider unit PRVS and/or the training unit TRS may be a real or virtual network of interconnected computers (e.g., a technical term for a real network is “cluster”, a technical term for a virtual network is “cloud”). The provider unit PRVS and/or the training unit TRS may also be embodied as a virtual system that is implemented on a real computer or a real or virtual network of interconnected computers (e.g., virtualization).

An interface IF and/or a training interface TIF may be a hardware or software interface (e.g., PCI bus, USB or Firewire). A computing unit CU and/or a training computing unit TCU may have hardware elements or software elements (e.g., a microprocessor or a field programmable gate array (FPGA)). A memory unit MU and/or a training memory unit TMU may be realized as a volatile working memory known as RAM (random access memory) or as a nonvolatile mass storage device (e.g., hard disk, USB stick, SD card, solid state disk (SSD)).

The interface IF and/or the training interface TIF may, for example, include a number of sub-interfaces that perform different acts of the respective methods. In other words, the interface IF and/or the training interface TIF may also be understood as a plurality of interfaces IF or as a plurality of training interfaces TIF. The computing unit CU and/or the training computing unit TCU may, for example, include a number of sub-computing units that perform different acts of the respective methods. In other words, the computing unit CU and/or the training computing unit TCU may also be understood as a plurality of computing units CU or as a plurality of training computing units TCU.

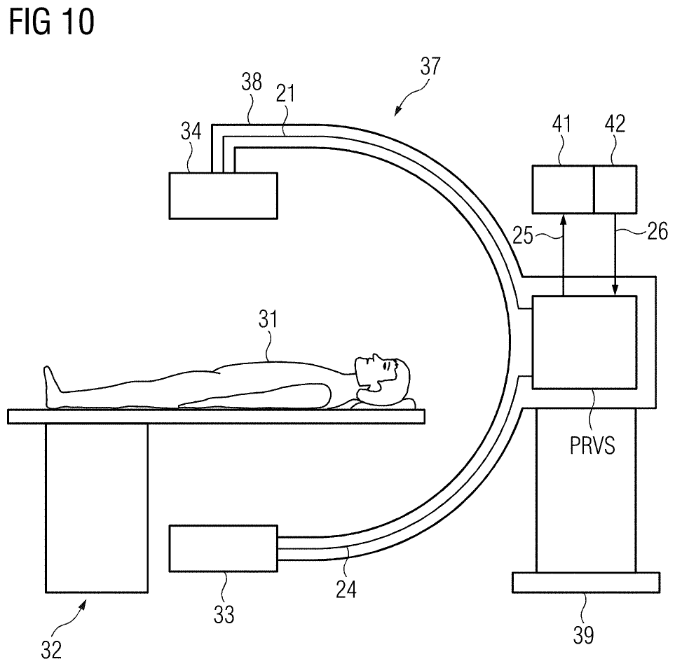

FIG. 10 schematically illustrates one embodiment of a medical C-arm X-ray apparatus 37, by way of example, for an embodiment of a medical imaging device. In this case, the medical C-arm X-ray apparatus 37 may include, for example, an embodiment of a provider unit PRVS for providing PROF-BFP a blood flow parameter set BFP for a vascular malformation MF. In this case, the medical imaging device 37 (e.g., the provider unit PRVS) is embodied for carrying out an embodiment of a computer-implemented method for providing PROV-BFP a blood flow parameter set BFP for a vascular malformation MF.

In this case, the medical C-arm X-ray apparatus 37 also includes a detector unit 34 and an X-ray source 33. In order to acquire the time-resolved image data BD, the arm 38 of the C-arm X-ray apparatus 37 may be mounted so as to be movable about one or more axes. The medical C-arm X-ray apparatus 37 may also include a movement device 39 that enables the C-arm X-ray apparatus 37 to move in space.

In order to acquire the time-resolved image data BD of the vessel section VS of the examination subject 31 arranged on a patient support and positioning device 32, the provider unit PRVS may send a signal 24 to the X-ray source 33. The X-ray source 33 may thereupon emit an X-ray beam (e.g., a cone beam and/or fan beam and/or parallel beam). When the X-ray beam, following an interaction with the vessel section VS of the examination subject 31 that is to be imaged, is incident on a surface of the detector unit 34, the detector unit 34 may send a signal 21 to the provider unit PRVS. The provider unit PRVS may receive REC-BD the time-resolved image data BD, for example, with the aid of the signal 21.

In addition, the medical C-arm X-ray apparatus 37 may include an input unit 42 (e.g., a keyboard) and/or a visualization unit 41 (e.g., a monitor and/or display). The input unit 42 may be integrated in the visualization unit 41 (e.g., in the case of a capacitive input display). This enables the medical C-arm X-ray apparatus 37 (e.g., the proposed computer-implemented method for providing PROV-BFP a blood flow parameter set BFP for a vascular malformation MF) to be controlled by an input by a member of the operating staff at the input unit 42. For this purpose, the input unit 42 may, for example, send a signal 26 to the provider unit PRVS.

The visualization unit 41 may also be embodied to display information and/or graphical representations of information of the medical imaging device 37 and/or the provider unit PRVS and/or further components. For this purpose, the provider unit PRVS may, for example, send a signal 25 to the visualization unit 41. For example, the visualization unit 41 may be embodied for displaying a graphical representation of the time-resolved image data BD and/or the image ABB of the vessel section VS and/or the vessel section model VM and/or the segmented vascular malformation MF and/or the three-dimensional pressure distribution and/or the blood flow parameter set. In one embodiment, a graphical (e.g., color-coded) representation of the image ABB of the vessel section VS and/or of the vessel section model VM and/or of the three-dimensional pressure distribution may be displayed on the visualization unit 41. The graphical representation of the image ABB of the vessel section VS and/or of the vessel section model VM and/or of the three-dimensional pressure distribution may also include an overlay (e.g., a weighted overlay).

The schematic views contained in the described figures do not depict a scale or proportions of any kind.

The methods described in detail in the foregoing, as well as the illustrated devices, are exemplary embodiments that may be modified in the most diverse ways by the person skilled in the art without departing from the scope of the invention. Further, the use of the indefinite articles “a” or “an” does not exclude the possibility that the features in question may also be present more than once. Similarly, the terms “unit” and “element” do not rule out the possibility that the components in question consist of a plurality of cooperating subcomponents, which, if necessary, may also be distributed in space.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A computer-implemented method for providing a blood flow parameter set for a vascular malformation, the computer-implemented method comprising:
receiving time-resolved image data, wherein the time-resolved image data maps a change over time in a vessel section of an examination subject, and wherein the vessel section includes the vascular malformation;
reconstructing a time-resolved image of the vessel section from the time-resolved image data;
segmenting the vascular malformation in the time-resolved image of the vessel section;
identifying at least one afferent vessel at the vascular malformation based on the time-resolved image of the vessel section;
identifying at least one efferent vessel at the vascular malformation based on the time-resolved image of the vessel section;
determining an average blood flow velocity parameter for each of the at least one afferent vessel and the at least one efferent vessel;
determining a vessel cross-sectional area parameter for each of the at least one afferent vessel and the at least one efferent vessel;
determining the blood flow parameter set for the vascular malformation based on the average blood flow velocity parameters and the vessel cross-sectional area parameters; and
providing the blood flow parameter set.

2. The computer-implemented method of claim 1, wherein the blood flow parameter set comprises at least one first blood flow parameter that corresponds to the at least one afferent vessel,
wherein the blood flow parameter set comprises at least one second blood flow parameter that corresponds to the at least one efferent vessel,
wherein the computer-implemented method further comprises comparing a sum of the at least one first blood flow parameter with a sum of the at least one second blood flow parameter, and
wherein the computer-implemented method is carried out repeatedly as of a predetermined discrepancy between the sums, starting with the identifying of the at least one afferent vessel.

3. The computer-implemented method of claim 1, further comprising:
determining a vessel section model based on the segmented vascular malformation, the determining of the vessel section model comprising adapting a volume mesh model;
determining a porosity parameter for the vascular malformation based on the vessel section model; and
determining a permeability parameter for the vascular malformation based on the vessel section model,
wherein determining the blood flow parameter set comprises determining a pressure ratio between the at least one afferent vessel and the at least one efferent vessel based on the porosity parameter, the permeability parameter, the average blood flow velocity parameters, and the vessel cross-sectional area parameters.

4. The computer-implemented method of claim 3, wherein determining the blood flow parameter set comprises applying a trained function to input data,
wherein the input data is based on the porosity parameter, the permeability parameter, the average blood flow velocity parameters, and the vessel cross-sectional area parameters, and
wherein at least one parameter of the trained function is based on a comparison between a training pressure ratio and a comparison pressure ratio.

5. The computer-implemented method of claim 4, wherein determining the blood flow parameter set further comprises determining a three-dimensional pressure distribution.

6. The computer-implemented method of claim 1, wherein the time-resolved image data maps a contrast medium bolus in the vessel section, and
wherein determining the average blood flow velocity parameter is based on a change in intensity over time in the time-resolved image of the vessel section due to the contrast medium bolus.

7. The computer-implemented method of claim 3, wherein the time-resolved image data maps a contrast medium bolus in the vessel section, and
wherein determining the average blood flow velocity parameter is based on a change in intensity over time in the time-resolved image of the vessel section due to the contrast medium bolus.

8. The computer-implemented method of claim 7, wherein the porosity parameter is determined based on a ratio between a volume of the vascular malformation and a volume of the contrast medium bolus within the vascular malformation.

9. The computer-implemented method of claim 7, wherein the time-resolved image of the vessel section has a number of voxels, and
wherein reconstructing a time-resolved image of the vessel section comprises assigning a bolus arrival time to each of the number of voxels in which the at least one afferent vessel, the at least one efferent vessel, the vascular malformation, or any combination thereof is imaged.

10. The computer-implemented method of claim 9, wherein identifying the at least one afferent vessel, identifying the at least one efferent vessel, or a combination thereof is based on a comparison of the bolus arrival time of different voxels of the time-resolved image of the vessel section.

11. The computer-implemented method of claim 1, wherein the blood flow parameter set includes a temporal blood volume flow parameter for each of the at least one afferent vessel and the at least one efferent vessel, and
wherein the temporal blood volume flow parameters are determined based on the respective average blood flow velocity parameter and the respective vessel cross-sectional area parameter.

12. A computer-implemented method for providing a trained function, the computer-implemented method comprising:
receiving average training blood flow velocity parameters, training vessel cross-sectional area parameters, and a segmented training vascular malformation, the receiving comprising applying a computer-implemented method for providing a blood flow parameter set for a vascular malformation, the computer-implemented method for providing the blood flow parameter set comprising:
receiving time-resolved image data, wherein the time-resolved image data maps a change over time in a vessel section of an examination subject, and wherein the vessel section includes the vascular malformation;
reconstructing a time-resolved image of the vessel section from the time-resolved image data;
segmenting the vascular malformation in the time-resolved image of the vessel section;
identifying at least one afferent vessel at the vascular malformation based on the time-resolved image of the vessel section;
identifying at least one efferent vessel at the vascular malformation based on the time-resolved image of the vessel section;
determining an average blood flow velocity parameter for each of the at least one afferent vessel and the at least one efferent vessel;
determining a vessel cross-sectional area parameter for each of the at least one afferent vessel and the at least one efferent vessel;
determining the blood flow parameter set for the vascular malformation based on the average blood flow velocity parameters and the vessel cross-sectional area parameters; and
providing the blood flow parameter set, wherein the average blood flow velocity parameters are provided as the average training blood flow velocity parameters, the vessel cross-sectional area parameters are provided as the training vessel cross-sectional area parameters, and the segmented vascular malformation is provided as the training vascular malformation;
determining a training vessel section model based on the training vascular malformation, the determining of the training vessel section model comprising adapting a volume mesh model;
determining a training porosity parameter for the training vascular malformation based on the training vessel section model;
determining a training permeability parameter for the training vascular malformation based on the training vessel section model;
determining a comparison pressure ratio between the at least one afferent vessel and the at least one efferent vessel based on the training porosity parameter, the training permeability parameter, the average training blood flow velocity parameters, and the training vessel cross-sectional area parameters;
determining a training pressure ratio between the at least one afferent vessel and the at least one efferent vessel, the determining of the training pressure ratio comprising applying the trained function to input data, wherein the input data is based on the training porosity parameter, the training permeability parameter, the average training blood flow velocity parameters, and the training vessel cross-sectional area parameters;
adjusting at least one parameter of the trained function based on a comparison between the training pressure ratio and the comparison pressure ratio; and
providing the trained function.

13. A medical imaging device comprising:
a processor configured to provide a blood flow parameter set for a vascular malformation, the provision of the blood flow parameter set comprising:
receipt of a time-resolved image data, wherein the time-resolved image data maps a change over time in a vessel section of an examination subject, and wherein the vessel section includes the vascular malformation;
reconstruction of a time-resolved image of the vessel section from the time-resolved image data;
segmentation of the vascular malformation in the time-resolved image of the vessel section;
identification of at least one afferent vessel at the vascular malformation based on the time-resolved image of the vessel section;
identification of at least one efferent vessel at the vascular malformation based on the time-resolved image of the vessel section;
determination of an average blood flow velocity parameter for each of the at least one afferent vessel and the at least one efferent vessel;
determination of a vessel cross-sectional area parameter for each of the at least one afferent vessel and the at least one efferent vessel;
determination of the blood flow parameter set for the vascular malformation based on the average blood flow velocity parameters and the vessel cross-sectional area parameters; and
provision of the blood flow parameter set,
wherein the medical imaging device is configured to acquire time-resolved image data, receive the time-resolved image data, provide the time-resolved image, or any combination thereof.
